(19) United States
(12) Patent Application Publication
(10) Pub. No.: US 2025/0066722 A1
(43) Pub. Date: Feb. 27, 2025

(54) METHODS FOR PRODUCTION OF FUNCTIONAL NEURONS

(71) Applicant: AXENT BIOSCIENCES INC., Oakland, CA (US)

(72) Inventor: Riya J. Muckom, Berkeley, CA (US)

(73) Assignee: AXENT BIOSCIENCES INC., Oakland, CA (US)

(21) Appl. No.: 18/703,641

(22) PCT Filed: Nov. 10, 2022
(86) PCT No.: PCT/US2022/079651

§ 371 Date: Apr. 22, 2024

Related U.S. Application Data
(60) Provisional application No. 63/278,902, filed on Nov. 12, 2021.

(51) Int. Cl. C12N 5/0793

(52) U.S. Cl. CPC C12N 5/0619; C12N 2501/13; C12N 2506/45; C12N 2513/00

(57) ABSTRACT

Provided herein is cell culture medium comprising a GDNF receptor RET agonist and its use for the production of a variety of functional neuron cell types from pluripotent cells.

### CROSS-REFERENCE TO RELATED APPLICATIONS

This application claims the benefit of U.S. Provisional Patent Application No. 63/278,902, filed Nov. 12, 2021, the full disclosure of which is incorporated herein by reference.

### BACKGROUND OF THE INVENTION

Human pluripotent stem cells (hPSCs), including embryonic and induced subtypes, are used extensively as source cells to derive functional terminal cell types, such as mature neurons, for a variety of applications including modeling of human development or disease¹, drug screening, and cell-based therapeutics. Derivation of functional cell types from hPSCs in vitro involves mimicking the natural development process of the precursor cell from a blastocyst environment whereby numerous biochemical signals such as growth factor morphogens and small molecules are presented to the cell in precise quantities, timing, and order to progressively specify the cell's fate.

Current methods for deriving functional neuronal subtypes from hPSCs in vitro rely heavily on the use of recombinant proteins supplemented in cell culture media to recapitulate the endogenous signaling process. For example, glial derived neurotrophic factor (GDNF) is prevalent in numerous protocols for neuronal differentiation from hPSCs to activate GDNF-mediated RET signaling that plays a crucial role in neuronal maintenance. Protocols to derive dopaminergic neuron precursors or neuroblasts from hPSCs that have shown efficacy in animal models of Parkinson's disease require addition of GDNF as often as 75% of the duration of the differentiation protocol. Production of hPSC-derived striatal neurons that have shown efficacy in animal models of Huntington's disease also requires substantial amounts of GDNF during the maturation phase of the differentiation process. Furthermore, the use of GDNF is prevalent in the production of several additional hPSC-derived neuronal subtypes as well, such as neuroepithelial stem cells, interneurons, cholinergic, and serotonin neurons that are cell therapy candidates for a variety of neurological disorders including stroke, neuropathic pain, schizophrenia, autism, epilepsy, and learning/memory deficits.

Extending beyond the cerebrum, cerebellar neurons that have been derived from hPSCs for modeling cerebellar degeneration and have potential for cerebellar degeneration therapy require substantial amounts of GDNF—in some cases more than 100 days of exposure—to reach a mature state. Peripheral sensory neurons, including nociceptors, mechanoreceptors, and proprioceptors, that were derived from hPSCs also heavily rely on use of GDNF to produce functional neurons for personalized neuropathy treatment modeling. Motor neuron differentiation and maturation from hPSCs for modeling or therapy of degenerative conditions including SMA and ALS require GDNF for nearly 50% of the differentiation time.

The majority of these neuronal cell types are candidates for cell replacement therapy for a range of neurological indications involving degeneration of neural tissue, however, manufacturing clinical grade cells for transplantation in humans has emerged as a significant bottleneck in translating these candidate therapies to the clinic. Depending on the indication, millions of cells are needed for a single patient dose which scales to an estimated manufacturing burden of up to 10¹⁴ cells per year for a single allogeneic product. For current Good Manufacturing Process (cGMP)-grade cells that are required for clinical development and commercialization of these cell therapy candidates, recombinant proteins are among the most costly raw materials. For example, in the 40-day protocol for production of dopaminergic neurons from hPSCs, GDNF, BDNF, and TGF-beta are supplemented into the cell culture media for 30 days, amounting to 50% of the total cost of reagents.

### SUMMARY OF THE INVENTION

Provided herein are in vitro methods for producing a variety of functional neuron cell types by culturing in a differentiation cell culture medium comprising a glial cell line-derived neurotrophic factor (GDNF) receptor RET (transmembrane receptor tyrosine kinase REarranged in Transfection) agonist, preferably wherein the differentiation cell culture medium is essentially free of proteins. In some aspects, a functional neuron cell is produced from an induced (stem cell-derived) neuronal precursor cell according to the methods herein described. In other aspects, a functional neuron cell is produced from a mammalian pluripotent cell according to the methods herein described.

Also provided herein is a cell culture medium useful for producing a variety of functional neuron cell types, wherein the cell culture medium comprises one or more GDNF receptor RET agonists. In preferred aspects, the cell culture medium does not comprise GNDF, BDNF (brain-derived neurotrophic factor) and TGFβ and preferably is essentially free of proteins.

### DESCRIPTION OF THE DRAWINGS

FIG. 1 illustrates a comparison between a standard protocol (i) for differentiation of hPSCs into dopaminergic neurons and the process described herein (ii). In contrast to the standard protocol, dopaminergic neurons are produced according to the present methods in induction media that does not contain FGF8 (during days 0-10) and in differentiation media that does not contain GDNF, BDNF or TGFβ3 (from day 11 to day 20+).

FIG. 2 illustrates periodic imaging of proliferating and differentiating neural aggregates in differentiation media containing BT-13 and DAPT and free of GDNF, BDNF and TGFβ3.

FIG. 3 illustrates immunocytochemistry staining for DAPI (blue) and tyrosine hydroxylase (TH) at 20 day of representative aggregates differentiated using differentiation media containing BT-13 and DAPT and free of GDNF, BDNF and TGFβ3.

FIG. 4 illustrates the results of a cost analysis comparing a standard protocol for differentiation of human pluripotent stem cells (hPSCs) into dopaminergic neurons to the present methods.

FIG. 5 illustrates tyrosine hydroxylase-positive neurons at day 18 produced by culturing hPSCs in induction media that does not contain FGF8 (during days 0-10) and in differentiation media containing 10 nM Q525 (and which is free of GDNF, BDNF and TGFβ3) (from day 11 to day 20+).

### DETAILED DESCRIPTION OF THE INVENTION

### Definitions

“Activators,” as used herein, refer to compounds that increase, induce, stimulate. activate, facilitate, or enhance activation the signaling function of the molecule or pathway, e.g., Wnt signaling, SHH signaling, etc.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of dopaminergic neurons, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “GDNF receptor RET agonist” encompasses molecules that indirectly or directly activate the transmembrane receptor tyrosine kinase RET. A GDNF receptor RET agonist may indirectly activate RET by increasing the activity of a GDNF family ligand (GFL) selected from glial cell line-derived neurotrophic factor (GDNF), artemin (ARTN), neurturin (NRTN) and persephin (PSPN), all of which signal through the transmembrane receptor tyrosine kinase RET. Direct activation of RET by a GDNF receptor RET agonist occurs independently of GFL proteins. By way of example, BT-13 and BT-18 activate RET directly, whereas X1B4035 indirectly activates RET by increasing the activity of GDNF or ARTN.

The term “dopaminergic neuron” is intended to encompass specifically it intends to include neuronal cells that express tyrosine hydroxylase and includes dopamine precursors and dopamine neuroblasts.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “undifferentiated” refers to a cell that has not yet developed into a specialized cell type.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of midbrain DA cells, or precursors thereof, such as EN-1, OTX2, TH, NURR1, FOXA2, and LLMX1A).

As used herein, the term “marker” or “cell marker” or “biomarker” refers to gene or protein that identifies a particular cell or cell type. A marker for a cell may not be limited to one marker, markers may refer to a “pattern” of markers such that a designated group of markers may identity a cell or cell type from another cell or cell type.

Provided herein is a use of a GDNF receptor RET agonist as a cell culture medium additive that obviates the need for GDNF, BDNF and TGFβ in the production of functional neurons from induced neuronal precursor cells or mammalian pluripotent cells.

In some aspects, an in vitro method for producing a neuron from an induced neuronal precursor cell (iNPC) is provided, the method comprising a step of culturing the iNPC in a differentiation cell culture medium comprising a GDNF receptor RET agonist. In some aspects, an iNPC for use according to the method expresses the markers Pax6, Nestin, and CD133.

In preferred embodiments, the GDNF receptor RET agonist is BT-13 (N,N-diethyl-3-[4-[4-fluoro-2-(trifluoromethyl)-benzoyl]piperazin-1-yl]-4-methoxybenzenesulfonamide) having the structure

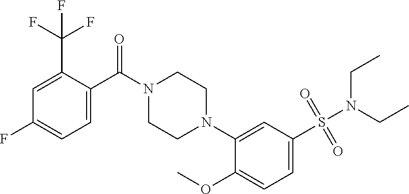 [SMILES: CCN(CC)S(=O)(=O)c1ccc(OC)c(N2CCN(C(=O)c3ccc(F)cc3C(F)(F)F)CC2)c1]

and/or BT-18 ([4-[5-[(6,7-dimethoxy-3,4-dihydro-1H-isoquinolin-2-yl)sulfonyl]-2-methoxyphenyl]piperazin-1-yl]-[4-fluoro-2-(trifluoromethyl)-phenyl]methanone) having the structure:

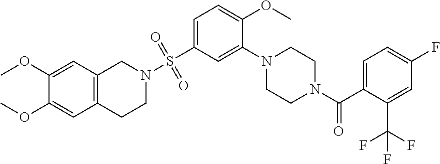 [SMILES: COc1cc2c(cc1OC)CN(S(=O)(=O)c1ccc(OC)c(N3CCN(C(=O)c4ccc(F)cc4C(F)(F)F)CC3)c1)CC2]

and/or BT-44. In some preferred embodiments, the differentiation cell culture medium comprises BT-13, BT-18 and/or BT-44 at a concentration of from about 2-20 μM. In other embodiments, the differentiation cell culture medium comprises from about 2 to 10 μM or about 5 μM BT-13, BT-18 and/or BT-44.

In related embodiments, the GDNF receptor RET agonist is XIB4035

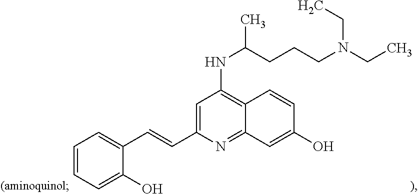 [SMILES: CCN(CC)CCCC(C)Nc1cc(/C=C/c2ccccc2O)nc2cc(O)ccc12]

preferably wherein the differentiation cell culture medium comprises from about 10-1000 nM XIB4035.

In other embodiments, the GDNF receptor RET agonist is Q525

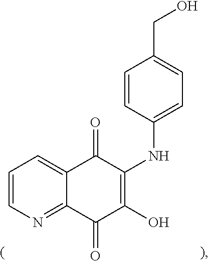 [SMILES: O=C1C(Nc2ccc(CO)cc2)=C(O)C(=O)c2ncccc21]

preferably wherein the differentiation cell culture medium comprises from about 1-100 nM Q525.

In other embodiments, the GDNF receptor RET agonist is selected from those listed at Table 3 of Jmaeffet al., JBC, 295(19):6532-6542 (2020), the structure of each of which is identified at Table 1 of that reference, the entire contents of which are incorporated herein by reference. In some preferred embodiments, the differentiation cell culture medium comprises from about 1-100 nM Q525 or Q508.

In other embodiments, the GDNF receptor RET agonist is dopamine neuron stimulating peptide-11 (DNSP-11; PPEAPAEDRSL (SEQ ID NO:1)).

In other embodiments, the GDNF receptor RET agonist is selected from among those described in Runeberg-Roos et al., Neurobiol. Dis., 96:335-345 (2016), Jmaeffet al., Mol. Pharmacol., 98:1-12 (2020), Mahato et al., Mov. Disord., 35:245-255 (2020), Sidorova et al., Front. Pharmacology, 8:365 (2017), and Sidorova et al., Int. J. Mol. Sci., 21(18), 6575 (2020), the entire contents of each of which is incorporated herein by reference. Other GDNF receptor RET agonists useful according to the present methods include those described in U.S. Pat. No. 8,901,129 (e.g. BT10, BT16, BT17 or BT292651), the entire contents of which are incorporated herein by reference.

In some preferred embodiments, the differentiation cell culture medium comprises a GDNF receptor RET agonist, e.g. BT-13 or Q525, and further comprises a notch pathway inhibitor such as DAPT (N-[2S-(3,5-difluorophenyl)acetyl]-L-alanyl-2-phenyl-1,1-dimethylethyl ester-glycine) and/or dibutyryl cAMP (db-cAMP), RO4929097, BMS-906024, YO-01027, LY-411575, or tangeretin. In some preferred embodiments, the differentiation cell culture medium comprises DAPT (e.g. at a concentration of 5 μM to 20 μM, preferably about 10 μM).

In some embodiments, the differentiation cell culture medium is free of serum and comprises (i) a neurobasal medium (e.g. Thermo Fisher Scientific, 11320033) optionally supplemented with glutamine (e.g. glutamax) and/or N2 supplement (e.g. Thermo Fisher Scientific, 17502048) and/or B27 supplement (e.g. Thermo Fisher Scientific, 17504044) and/or ascorbic acid (ii) a GDNF receptor RET agonist, e.g. BT13 or Q525, and (iii) a notch pathway inhibitor, preferably DAPT and/or db-cAMP. The presence of ascorbic acid is not required to produce functional neurons according to the present methods, but may contribute to cell health and maintenance.

In some aspects, B-27 and N-2 supplements in the differentiation cell culture medium are replaced with one or more insulin receptor activator molecules, preferably a selective insulin receptor activator such as demethylasterriquinone B1 (DMAQ-B1 aka DAQB1), preferably at a concentration between 10-100 μM, or 5,8-diacetyloxy-2,3-dichloro-1,4-naphthoquinone (DDN). Thus, in some preferred embodiments, the differentiation cell culture medium is serum-free, and comprises (i) a neurobasal medium (e.g. Thermo Fisher Scientific, 11320033) (ii) an insulin receptor activator, preferably DAQB1 and (iii) a GDNF receptor RET agonist, e.g. BT13 or Q525, and (iv) a notch pathway inhibitor, preferably DAPT and/or db-cAMP, wherein the medium does not comprise B-27 supplement and N-2 supplement. Optionally, the differentiation cell culture medium comprises an iron transport molecule such as hinokitiol (5-50 μM) and/or an alternative to BSA such as recombinant human serum albumin (HSA) (10-100 μg/mL). In some aspects, the differentiation cell culture medium is a fully chemically defined serum-free and xeno-free media (e.g. CTS KnockOut SR XenoFree supplement (12618012)). By xeno-free it is intended that the culture medium does not contain bovine or other non-human, animal-derived components.

In particularly preferred embodiments, the differentiation cell culture medium does not comprise one or more of GDNF, BDNF and TGFβ. In particularly preferred embodiments, the differentiation cell culture medium is substantially free of GDNF, BDNF and TGFβ. In other preferred embodiments, the differentiation cell culture medium is essentially free of proteins.

In some aspects, an induced neuronal precursor cell for use according to the methods described herein is obtained by culturing a mammalian pluripotent cell in a neural induction medium for a time suitable to produce the induced neuronal precursor cell. Typically, pluripotent cells express the following markers: Oct4, SOX2, Nanog, SSEA3, SSEA4, TRA 1/81.

In some embodiments, the pluripotent cells are human pluripotent cells. In another embodiment, the pluripotent cells are non-human mammalian pluripotent cells. In preferred embodiments, the pluripotent cells are stem cells. In some aspects, the stem cells are embryonic stem cells, preferably human embryonic stem cells (e.g. human embryonic stem cell lines SA01, VUB01, HUES 24, H1, H9, WT3, HUES1). In other aspects the stem cells are non-human (e.g. mouse, rodent or primate) embryonic stem cells. In other aspects, the stem cells are adult human stem cells. In other preferred embodiments, the stem cells are induced pluripotent stem cells (iPSC). Induced pluripotent stem cells are a type of pluripotent stem cells artificially derived from a non-pluripotent, typically adult somatic cell, by inducing a forced expression of certain genes. For example, human dermal fibroblasts can be reprogrammed into pluripotent stem cells using the four Yamanaka factors (Oct3/4, Sox2, Klf4 and cMyc). See e.g. Takahashi K, Yamanaka S., Cell. 2006; 126(4):663-676, the entire contents of which are incorporated herein by reference.

In some aspects, to produce an induced neuronal precursor cell from a mammalian pluripotent stem cell, neuronal induction of the pluripotent stem cell is initiated by culturing the stem cell in the presence of dual inhibitors of the SMAD pathway (generally by inhibiting the bone morphogenetic protein (BMP) and TGFβ signaling pathways), without the need for feeder cells.

Culturing a stem cell in the presence of a BMP inhibitor encompasses any culture condition capable of inhibiting the BMP signaling pathway, whether by directly acting on BMPs and their receptors or by inhibiting their expression. Suitable inhibitors of the BMP signaling pathway include, without limitation LDN193189, DMH1, Noggin, Chordin, Follistatin, Dorsomorphin (6-[4-(2-Piperidin-1-yl-ethoxy)phenyl]-3-pyridin-4-yl-pyrazolo [1,5-a]pyrimidine), K02288, LDN212854, and ML347, LDN214117. In preferred embodiments, the BMP inhibitor is LDN193189. The concentration of BMP inhibitor in the culture is a concentration effective to inhibit the BMP signaling pathway.

Culturing a stem cell in the presence of a TGFβ inhibitor encompasses any culture condition capable of inhibiting TGFβ, whether by directly acting on TGFβ to inhibit its function or by inhibiting production of TGFβ per se. Suitable inhibitors of the TGFβ signaling pathway include, without limitation, A83-01, SB-431542, LY364947, SB-525334, SD208, LY2157299, LY2109761, SB-505124, GW788388 and EW-7197. In preferred embodiments, the TGFβ inhibitor is SB-431542. The concentration of TGFβ inhibitor in the culture is a concentration effective to inhibit TGF3.

In a preferred embodiment, the induced neuronal precursor cell is a floor pate-based progenitor cell, e.g. a midbrain floor plate cell. In some embodiments, for patterning to a midbrain fate, stem cells are cultured in induction medium comprising inhibitors of SMAD pathway and (i) an activator of sonic hedgehog (SHH) (ii) an activator of WNT signaling pathway and optionally (iii) an FGF receptor (FGFR) agonist. Representative methods for generating midbrain precursors include the methods described in U.S. Pat. No. 10,858,625, the entire contents of which are incorporated herein by reference.

The term “sonic hedgehog agonist” or “SHH agonist” as used herein includes recombinant sonic hedgehog, purmorphamine and SAG, which stands for Smoothened Agonist and is a chlorobenzothiophene-containing compound. Shh can also be replaced with recombinant mammalian Desert hedge hog (Dhh) or recombinant mammalian Indian hedge hog (Ihh). Activates Smoothened (SMO) can also be used. In preferred embodiments, the SHH activator is SAG.

Suitable activators of the WNT signaling pathway include GSK-3β inhibitors such CHIR99021, LiCl, BIO((2′Z,3′E)-6-Bromoindirubin-3′-oxime), Kenpaullone, A1070722, SB216763, CHIR98014, TWS119, Tideglusib, SB415286, Bikinin, IM-12, 1-Azakenpaullone, LY2090314, AZD1080, AZD2858, AR-A014418, TDZD-8, and Indirubin. In preferred embodiments, the WNT activator is CHIR99021.

An “FGF receptor (FGFR) agonist” as used herein means a molecule that can activate FGFR (e.g. molecules that bind to FGFR and induce the dimerization of the receptor and activate the signaling P13K pathway and Ras/ERK pathway). Nonlimiting examples of FGFR agonists include FGF2, FGF8 and SUN11602. In preferred embodiments, the FGFR agonist is FGF8 (e.g. recombinantly produced FGF8).

In some preferred aspects, the neural induction medium comprises (i) LDN193189 (LDN) to inhibit BMP signaling (ii) SB-431542 (SB) to inhibit TGFβ signaling (e.g. 10 mM) (iii) recombinant FGF8 (iv) smoothened agonist (SAG; 3-chloro-N-[trans-4-(methylamino)cyclohexyl]-N-[3-(pyridin-4-yl)benzyl]-1-benzothiophene-2-carboxamide) to activate sonic hedgehog signaling and (iv) CHIR99021 (CHIR, e.g. 10 mM) to activate WNT signaling. Alternatively, CT99021 (a GSK3 inhibitor) may be used to activate WNT signaling.

In other aspects, the neural induction medium comprises (i) LDN193189 (LDN) to inhibit BMP signaling (ii) smoothened agonist (SAG; 3-chloro-N-[trans-4-(methylamino)cyclohexyl]-N-[3-(pyridin-4-yl)benzyl]-1-benzothiophene-2-carboxamide) to activate sonic hedgehog signaling and (iii) CHIR99021 (CHIR, e.g. 10 mM) to activate WNT signaling, wherein the neural induction medium does not comprise an inhibitor of TGFβ signaling (such as SB-431542 (SB)) and/or wherein the neural induction medium does not comprise FGF8. In some aspects, an iPSC or ESC is identified by expression of Oct4/POU5F1, Nanog and Sox2.

In some aspects, a neural precursor cell is identified by expression of Pax6, Nestin and CD133.

In some aspects a midbrain progenitor cell is identified by expression of one or more (e.g. all) of the following markers: FOXA2, LMXIA, OXT2, EN1/2, GBX2, Wnt1, CNPY1, SPRY1, and Pax8.

In some aspects, a mature dopaminergic neuron is identified by expression of expression of one or more (e.g. all) of the following markers: tyrosine hydroxylase, COR1N, Nurr1, GRK2, Pitx3, DAT, LRTM1, ALCAM, DRD2, DBH, CHRNB3.

In some aspects, a method for producing a differentiated neuronal cell from a mammalian stem cell is provided comprising (i) culturing the stem cell in a neural induction medium, wherein the culturing results in the production of an induced neuronal precursor cell and (ii) culturing the induced neuronal precursor cell in a differentiation medium comprising GDNF receptor RET agonist, preferably BT13, wherein the culturing results in the production of a population of differentiated neuronal cells. In some embodiments, the stem cell is cultured in a neural induction medium according to step (i) for about 10-12 days, 9-11 days or about 10 days.

Advantageously, production of functional neurons from mammalian stem cells according to the present method does not require the presence of an FGFR agonist such as FGF8 in the neural induction medium of step (i). Accordingly, in some preferred embodiments, a method for producing a neuronal cell from a mammalian stem cell is provided comprising (i) culturing the stem cell in a neural induction medium comprising (a) an inhibitor of BMP signaling (b) an inhibitor of TGFβ signaling (c) an activator of sonic hedgehog (SHH) and (d) an activator of WNT, wherein the neural induction medium does not comprise an FGFR agonist and (ii) culturing the neuronal precursor cell in a differentiation medium comprising a GDNF receptor RET agonist, preferably BT13, whereby a neuronal cell is produced.

In other preferred embodiments, production of functional neurons from mammalian stem cells according to the present method is performed without including an inhibitor of TGFβ signaling in the neural induction medium of step (i). Accordingly, a method for producing a neuronal cell from a mammalian stem cell is provided comprising (i) culturing the stem cell in a neural induction medium comprising (a) an inhibitor of BMP signaling (b) an activator of sonic hedgehog (SHH) and (c) an activator of WNT, wherein the neural induction medium does not comprise an inhibitor of TGFβ signaling and (ii) culturing the neuronal precursor cell in a differentiation medium comprising a GDNF receptor RET agonist (e.g. BT13 or Q525), whereby a neuronal cell is produced. In related embodiments, the neural induction medium of step (i) does comprise an inhibitor of TGFβ signaling and does not comprise an FGFR agonist.

A variety of differentiated neuronal cells may be produced according to the present methods e.g. by varying the number of days the neural precursor cell is cultured in differentiation medium comprising a GDNF receptor RET agonist, e.g. BT13 or Q525, according to step (ii). In some aspects, a differentiated neuronal cell produced according to the methods described herein is selected from a dopamine precursor, a dopamine neuroblast, a striatal neuron, a neuroepithelial stem cell, a GABAergic interneuron, cortical interneurons, a cholinergic neuron, a serotonin interneuron, a cerebellar neuron, a sensory neuron, and a motor neuron.

Table 1 below illustrates different neuronal cell types that can be produced from mammalian pluripotent cells according to the present methods along with their potential medical use, the number of days of GDNF required to produce each cell type according to prior art methods, makers for identifying the neuronal cell type, and references describing the prior art methods, the contents of each of which is incorporated herein by reference. For each of the protocols below, a GDNF receptor RET agonist (e.g. BTJ3 or Q525) is substituted for GDNF (and for BDNF, TGFβ3 and other proteins depending on the protocol) according to the present methods.

TABLE 1

| | | | Days of | |
|---|---|---|---|---|
| | | | GDNF | |
| | | | out of total | |
| Cell Type | Markers | Indication/Application | protocol | reference |
| Dopamine | FOXA2, | Parkinson's Disease | 5/16 | Kirkeby, et al., Cell Rep. 1, 703-714 |
| precursors | LMX1A, | | | (2012), the entire contents of which are |
| | EN1, | | | incorporated herein by reference |
| | SPRY1, | | 9/20 | Song et al., J. Clin. Invest., 130: 904-920 |
| | WNT1, | | | (2020), the entire contents of which are |
| | CNPY1, | | | incorporated herein by reference |
| | PAX8 | | 15/25 | Adil et al., Sci. Rep. 7, 40573 (2017), the |
| | | | | entire contents of which are incorporated |
| | | | | herein by reference |
| | | | 16/28 | Doi et al., Nat. Commun. 11: 1-14 (2020), |
| | | | | the entire contents of which are |
| | | | | incorporated herein by reference |
| Dopamine | DRD2, | Parkinson's Disease | 13/45 | Chen et al., Cell Stem Cell, 18: 817-826 |
| neuroblasts | DBH, GBX2, | | | (2016), the entire contents of which are |
| | PITX3, | | | incorporated herein by reference |
| | CHRNB3, | | 14/25 | Kriks et al., Nature 480: 547-551 (2012), |
| | DAT, GRK2, | | | the entire contents of which are |
| | CORIN, | | | incorporated herein by reference |
| | LRTM1, | | 21/49 | Hallett et al., Cell Stem Cell, 16: 269-274 |
| | ALCAM | | | (2015), the entire contents of which are |
| | | | | incorporated herein by reference |
| | | | 30/40 | Adil et al., Sci, Rep. 7, 40573 (2017), the |
| | | | | entire contents of which are incorporated |
| | | | | herein by reference |
| Striatal | DARPP32, | Huntington's Disease | 11/36 | Arber C et al., Dev. 142: 1375-1386 |
| Neurons | CTIP2, | | | (2015), the entire contents of which are |
| | CALBINDIN, | | | incorporated herein by reference |
| | GABA | | 15/47 | Ma et al., Cell Stem Cell, 10: 455-464 |
| | | | | (2012), the entire contents of which are |
| | | | | incorporated herein by reference |
| | | | 20/35 | Adil et al., Stem Cell Reports 10: 1481- |
| | | | | 1491 (2018), the entire contents of which |
| | | | | are incorporated herein by reference |
| | | | 21/37 | Comella-Bolla et al., Mol. Neurobiol., |
| | | | | doi: 10.1007/s12035-020-01907-4, the |
| | | | | entire contents of which are incorporated |
| | | | | herein by reference |
| | | | 30/45 | Carri et al., Dev. 140: 301-312 (2013), the |
| | | | | entire contents of which are incorporated |
| | | | | herein by reference |
| Neuroepithelial | | Stroke | 14/24 | Tornero et al., Brain 136: 3561-3577 |
| Stem Cells | | | | (2013), the entire contents of which are |
| | | | | incorporated herein by reference |
| GABAergic | | Neuropathic pain | 4/25 | Manion et al., Pain |
| Interneuron | | | | doi: 10.1097/j.pain.0000000000001733, the |
| | | | | entire contents of which are incorporated |
| | | | | herein by reference |
| Cortical | | Schizophrenia, | 7/28 | Ni et al., Mol. Ther. - Methods Clin. Dev. 0, |
| Interneurons | | autism, and epilepsy | | (2019), the entire contents of which are |
| | | | | incorporated herein by reference |
| | | | 14/25 | Maroof et al., Cell Stem Cell 12: 559-572 |
| | | | | (2013), the entire contents of which are |
| | | | | incorporated herein by reference |
| | | | 14/35 | Kim et al., Stem Cells, 32: 1780-1804 |
| | | | | (2014), the entire contents of which are |
| | | | | incorporated herein by reference |
| Cholinergic | | Learning/memory | 7/35 | Liu et al., Nat. Biotechnol. 31: 440-447 |
| Neurons | | deficits | | (2013), the entire contents of which are |
| | | | | incorporated herein by reference |
| Serotonin | | Psychiatric disorders | 7/28 | Lu et al., Nat. Biotechnol., 34: 89-94 |
| Neurons | | | | (2016), the entire contents of which are |
| | | | | incorporated herein by reference |
| Cerebellar | | Cerebellar | 110/145 | Silva et al., Front. Bioeng. Biotechnol, 8, |
| Neurons | | degeneration | | 70 (2020), the entire contents of which are |
| | | | | incorporated herein by reference |
| Sensory | | Neuropathy Modeling | 37/50 | Salto-Diaz et al., Stem Cell Reports 0, |
| Neurons | | | | (2021), the entire contents of which are |
| | | | | incorporated herein by reference |
| | | | 20/30 | Chambers SM et al., Nat. Biotechnol., |
| | | | | 30: 715-720 (2012), the entire contents of |
| | | | | which are incorporated herein by reference |
| Motor Neurons | | SMA, ALS | 35/60 | Faravelli et al., Stem Cell Research and |
| | | | | Therapy, vol. 5, 87 (2014), the entire |
| | | | | contents of which are incorporated herein |
| | | | | by reference |

The type of neuron produced according to the method may be identified by expression of one or more surface markers. In some aspects, a dopaminergic neuron is produced. Dopaminergic neurons can be identified by expression of tyrosine hydroxylase and optionally one or more of DAT, COR1N, GIRK2, PITX3 and NURR1. In other aspects, a striatal neuron is produced. Striatal neurons can be identified by expression of one or more of DARPP32, CITP2, CALBINDIN, and GABA.

The capacity to generate action potentials is a hallmark of neuronal maturation and function, with different neuronal phenotypes exhibiting distinct, specific firing patterns. As such, functionality of a neuron produced according to the present methods may be confirmed, in addition to expression of one or more surface markers, by assessing the ability of the neuron to fire action potentials, e.g. using patch-clamp electrophysiology or using the voltage imaging methods described in Adil, M. et al., Sci. Rep. 7, 40573 (2017), the entire contents of which are incorporated herein by reference. Functional midbrain dopaminergic neurons, e.g., exhibit a firing pattern of periodic spikes at 2-5 Hz. Neurons produced according to the present methods may also be implanted (e.g. striatally) into an animal model, e.g. a Fisher 344 rat and survival of the grafted neurons assessed at a subsequent time point (e.g. 6 weeks post-implantation).

In some embodiments (e.g. utilizing embryoid body-derived neurosphere-based induction), following step (i), cell clusters are dissociated to single cells (e.g. on day 11) for culturing in differentiation medium according to step (ii). In other embodiments a 2-dimensional monolayer-based method (e.g. a 2D Matrigel-coated surface) is employed. In other embodiments, a 3-dimensional cell culture-based method is employed, e.g. in which cells are embedded in a biomaterial such as alginate, collagen, hyaluronic acid or a material as described in Adil, M. et al., Sci. Rep. 7, 40573 (2017), the entire contents of which are incorporated herein by reference.

In some aspects, a step of culturing in differentiation medium as herein described occurs for a period of time sufficient to produce the desired neuron (see e.g. Table 1). Generally, culturing in differentiation medium as herein described occurs for a period of from about 4 days to about 110 days. In some aspects, culturing in differentiation medium as herein described occurs for a period of about 4 to about 60 days, or about 5 days to about 40 days or about 16 days to 32 days.

In related aspects, a cell population produced according to the present methods is provided. Cell populations produced according to the present methods typically may comprise other cell types in addition to differentiated neuronal cells. In one embodiment, the populations of the invention are characterized in that they comprise at least 10%, 20%, 30%, 40%, 50%, 60%, 70%, 80% and preferably at least 90% or at least 95% of cells that exhibit high expression of at least one biomarker characteristic of a differentiated neuron, for example TH gene product.

Other biomarkers characteristic of differentiated neuron cells depends on the type of neuron produced, but include without limitation, one or more of the markers listed at Table 1.

Any methods known in the art for measuring gene expression may be used, in particular, quantitative methods such as, real time quantitative PCR or microarrays, or methods using gene reporter expression or qualitative methods such as immunostaining or cell sorting methods identifying cells exhibiting specific biomarkers, including cell surface markers.

In some aspects, a cell culture differentiation medium useful for producing a functional neuron from a mammalian pluripotent stem cell or from an induced neuronal precursor cell is provided, the differentiation medium comprising a GDNF receptor RET agonist, e.g. BT-13 or Q525. In some embodiments, the differentiation medium is useful for producing a tyrosine hydroxylase-positive dopaminergic neuron. In some embodiments, the culture medium further comprises a notch pathway inhibitor (e.g. DAPT and/or db-cAMP). In preferred embodiments, the culture medium does not contain GDNF, BDNF and TGF-β. In other preferred embodiments, the culture medium is essentially free of proteins. In some aspects, the cell culture medium comprises a neurobasal medium supplemented with N2 supplement and B17 supplement. In other aspects, the cell culture medium comprises a neurobasal medium and an insulin receptor activator, preferably DMAQ-B1, and does not comprise N2 supplement and does not comprise B17 supplement.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

### Methods

Human Pluripotent Stem Cell Culture. Human induced pluripotent stem cells (hPSCs) (ThermoFisher A 18945) were subcultured in monolayer format on a layer of 1% Matrigel and maintained in Essential 8 medium during expansion. At 80% confluency, H9s were passaged using Versene solution and replated at a 1:8 split.

3D hPSC Culture Seeding. hPSCs were dissociated into single cells using Accutase solution and resuspended in Essential 8 (E8) medium containing 10 μM Y-27632 (Rock Inhibitor, RI). hPSCs were counted and resuspended at defined densities in 11% AXgel on ice. Cells suspended in AXgel were dispensed into a multi-well tissue culture plate and heated to 37° C. for 15 minutes and afterward pre-warmed E8 medium containing 10 μM RI was added to each well. 3D cell suspensions remained in E8 with RI for 2 days (from Day −2 to Day 0).

3D Dopaminergic Neuron Differentiation. Starting Day 0, hPSCs in AXgel were transitioned to differentiation media to induce neural lineage commitment and subsequently specification into midbrain dopaminergic neurons. Starting Day 11, neural precursors were transitioned to maturation media containing GDNF agonist BT-13 (5 μM) or Q525 (5 nM) in place of proteins GDNF, BDNF, and TGF-β. Media formulations were according to Table 2:

TABLE 2
BT-13 or Q525 Substitution Media for Differentiation of hPSCs to DA Neurons

| | DMEM/ | Neuro- | | Gluta- | | | | | | | BT-13/ |
|---|---|---|---|---|---|---|---|---|---|---|---|
| Day | F12 | basal | P/S | max | B-27 | N-2 | LDN | CHIR | SAG | DAPT | Q525 |
| 0 | 50% | 50% | 0.50% | 1:100 | 1:50 | 1:100 | 100 nM | 0 | 0 | 0 | 0 |
| 1 | 50% | 50% | 0.50% | 1:100 | 1:50 | 1:100 | 100 nM | 0 | 5 uM | 0 | 0 |
| 2 | 50% | 50% | 0.50% | 1:100 | 1:50 | 1:100 | 100 nM | 0 | 5 uM | 0 | 0 |
| 3 | 50% | 50% | 0.50% | 1:100 | 1:50 | 1:100 | 100 nM | 10 uM | 5 uM | 0 | 0 |
| 4 | 50% | 50% | 0.50% | 1:100 | 1:50 | 1:200 | 100 nM | 10 uM | 5 uM | 0 | 0 |
| 5 | 50% | 50% | 0.50% | 1:100 | 1:50 | 1:200 | 100 nM | 10 uM | 5 uM | 0 | 0 |
| 6 | 50% | 50% | 0.50% | 1:100 | 1:50 | 1:200 | 100 nM | 10 uM | 0 | 0 | 0 |
| 7 | 50% | 50% | 0.50% | 1:100 | 1:50 | 1:200 | 100 nM | 10 uM | 0 | 0 | 0 |
| 8 | 50% | 50% | 0.50% | 1:100 | 1:50 | 1:200 | 100 nM | 10 uM | 0 | 0 | 0 |
| 9 | 50% | 50% | 0.50% | 1:100 | 1:50 | 1:200 | 100 nM | 10 uM | 0 | 0 | 0 |
| 10 | 50% | 50% | 0.50% | 1:100 | 1:50 | 1:200 | 100 nM | 10 uM | 0 | 0 | 0 |
| 11 | 0% | 100% | 0.50% | 1:100 | 1:100 | 1:200 | 0 | 0 | 0 | 10 uM | 5 uM/5 nM |
| 12 | 0% | 100% | 0.50% | 1:100 | 1:100 | 1:200 | 0 | 0 | 0 | 10 uM | 5 uM/5 nM |
| 13 | 0% | 100% | 0.50% | 1:100 | 1:100 | 1:200 | 0 | 0 | 0 | 10 uM | 5 uM/5 nM |
| 14 | 0% | 100% | 0.50% | 1:100 | 1:100 | 1:200 | 0 | 0 | 0 | 10 uM | 5 uM/5 nM |
| 15 | 0% | 100% | 0.50% | 1:100 | 1:100 | 1:200 | 0 | 0 | 0 | 10 uM | 5 uM/5 nM |
| 16 | 0% | 100% | 0.50% | 1:100 | 1:100 | 1:200 | 0 | 0 | 0 | 10 uM | 5 uM/5 nM |
| 17 | 0% | 100% | 0.50% | 1:100 | 1:100 | 1:200 | 0 | 0 | 0 | 10 uM | 5 uM/5 nM |
| 18 | 0% | 100% | 0.50% | 1:100 | 1:100 | 1:200 | 0 | 0 | 0 | 10 uM | 5 uM/5 nM |
| 19 | 0% | 100% | 0.50% | 1:100 | 1:100 | 1:200 | 0 | 0 | 0 | 10 uM | 5 uM/5 nM |
| 20 | 0% | 100% | 0.50% | 1:100 | 1:100 | 1:200 | 0 | 0 | 0 | 10 uM | 5 uM/5 nM |
| 21 | 0% | 100% | 0.50% | 1:100 | 1:100 | 1:200 | 0 | 0 | 0 | 10 uM | 5 uM/5 nM |
| 22 | 0% | 100% | 0.50% | 1:100 | 1:100 | 1:200 | 0 | 0 | 0 | 10 uM | 5 uM/5 nM |
| 23 | 0% | 100% | 0.50% | 1:100 | 1:100 | 1:200 | 0 | 0 | 0 | 10 uM | 5 uM/5 nM |
| 24 | 0% | 100% | 0.50% | 1:100 | 1:100 | 1:200 | 0 | 0 | 0 | 10 uM | 5 uM/5 nM |
| 25 | 0% | 100% | 0.50% | 1:100 | 1:100 | 1:200 | 0 | 0 | 0 | 10 uM | 5 uM/5 nM |
| 26 | 0% | 100% | 0.50% | 1:100 | 1:100 | 1:200 | 0 | 0 | 0 | 10 uM | S uM/5 nM |
| 27 | 0% | 100% | 0.50% | 1:100 | 1:100 | 1:200 | 0 | 0 | 0 | 10 uM | 5 uM/5 nM |
| 28 | 0% | 100% | 0.50% | 1:100 | 1:100 | 1:200 | 0 | 0 | 0 | 10 uM | 5 uM/5 nM |
| 29 | 0% | 100% | 0.50% | 1:100 | 1:100 | 1:200 | 0 | 0 | 0 | 10 uM | 5 uM/5 nM |
| 30 | 0% | 100% | 0.50% | 1:100 | 1:100 | 1:200 | 0 | 0 | 0 | 10 uM | 5 uM/5 nM |
| 31 | 0% | 100% | 0.50% | 1:100 | 1:100 | 1:200 | 0 | 0 | 0 | 10 uM | 5 uM/5 nM |
| 32 | 0% | 100% | 0.50% | 1:100 | 1:100 | 1:200 | 0 | 0 | 0 | 10 uM | 5 uM/5 nM |
| 33 | 0% | 100% | 0.50% | 1:100 | 1:100 | 1:200 | 0 | 0 | 0 | 10 uM | 5 uM/5 nM |
| 34 | 0% | 100% | 0.50% | 1:100 | 1:100 | 1:200 | 0 | 0 | 0 | 10 uM | 5 uM/5 nM |
| 35 | 0% | 100% | 0.50% | 1:100 | 1:100 | 1:200 | 0 | 0 | 0 | 10 uM | 5 uM/5 nM |
| 36 | 0% | 100% | 0.50% | 1:100 | 1:100 | 1:200 | 0 | 0 | 0 | 10 uM | 5 uM/5 nM |
| 37 | 0% | 100% | 0.50% | 1:100 | 1:100 | 1:200 | 0 | 0 | 0 | 10 uM | 5 uM/5 nM |
| 38 | 0% | 100% | 0.50% | 1:100 | 1:100 | 1:200 | 0 | 0 | 0 | 10 uM | 5 uM/5 nM |
| 39 | 0% | 100% | 0.50% | 1:100 | 1:100 | 1:200 | 0 | 0 | 0 | 10 uM | 5 uM/5 nM |
| 40 | 0% | 100% | 0.50% | 1:100 | 1:100 | 1:200 | 0 | 0 | 0 | 10 uM | 5 uM/5 nM |

Additional reagents are listed in Table 3:

TABLE 3
Additional Reagent Details

| Cell Culture | | |
|---|---|---|
| Reagents | Concentration | Manufacturer, Cat. No |
| Y-27632 (Rock | 10 μM | SelleckChem, S1049 |
| Inhibitor) | | |
| DMSO | 100% | Sigma, D2650-5X5ML |
| Accutase Solution | 100% | Life Technologies, |
| | | A11105-01 |
| Versene Solution | 100% | ThermoFisher, |
| | | 15040066 |
| Essential-8 Media | 100% | Life Technologies, |
| | | A1517001 |
| hESC-qualified | 1% | Corning, 354277 |
| Matrigel | | |
| Penicillin/Streptomycin | 0.5% | ThermoFisher, |
| | | 15140122 |
| Hoechst | 1:2000 | Life Technologies, |
| | | H3570 |
| Mouse anti-Tyrosine | 1:1000 | Pel-Freez, P40101-150 |
| Hydroxylase | | |
| Donkey anti-Rabbit Cy3 | 1:250 | Jackson, 711-165-152 |
| Donkey anti-Rabbit 647 | 1:250 | Jackson, 711-605-152 |
| Donkey anti-Mouse 488 | 1:250 | Jackson, 711-545-152 |
| Donkey Serum | 5% | Sigma, D9663-10ML |
| Triton X-100 | 0.25% | Sigma, X100-100mL |
| Paraformaldehyde | 4% in PBS | SCBT, sc-281692 |

Immunocytochemistry. At the endpoint of the experiment, cell aggregates were harvested from AXgel by replacing warm media with chilled media and plated on 8-chamber culture slides coated with 10 μg/mL laminin and cultured overnight in the incubator to allow aggregates to adhere to the surface. Aggregates attached to the culture slide were then fixed using 4% paraformaldehyde (PFA) for 15 minutes. Aggregates were washed twice in PBS for 5 minutes each and incubated in 0.25% Triton-X+5% donkey serum in PBS for 10 minutes to permeabilize cells. After permeabilization, aggregates were washed 5 times in 5% donkey serum for 5 minutes each and incubated with primary antibodies of interest diluted in PBS+donkey serum (dilution details in Table 3), and stored overnight at 4° C. After primary staining, aggregates were washed twice in PBS for 5 minutes each and incubated in solution containing the corresponding secondary antibodies (dilution details in Table 3), and incubated at 37° C. for 2 hours. After secondary staining, aggregates were washed twice in PBS for 5 minutes each and culture slides were mounted with a cover slip to be imaged.

Microscopy. Live cell aggregates suspended in AXgel were periodically imaged during the experiment using an EVOS XL Core Imaging System for transmitted light microscopy available in the Cell and Tissue Analysis Facility through QB3-Berkeley. Fixed, stained, and mounted cell aggregates were imaged with a 20× or 40× objective using a Perkin Elmer Opera Phenix automated confocal fluorescence microscope available in the High-Throughput Screening Facility through QB3-Berkeley. Laser exposure time and power was kept constant for a fluorescence channel within an imaging set.

### Results and Discussion

Replacement of GDNF, BDNF, and TGF-β with BT-13 or Q525 is sufficient to produce hPSC-derived neurons expressing the functional biomarker Tyrosine Hydroxylase.

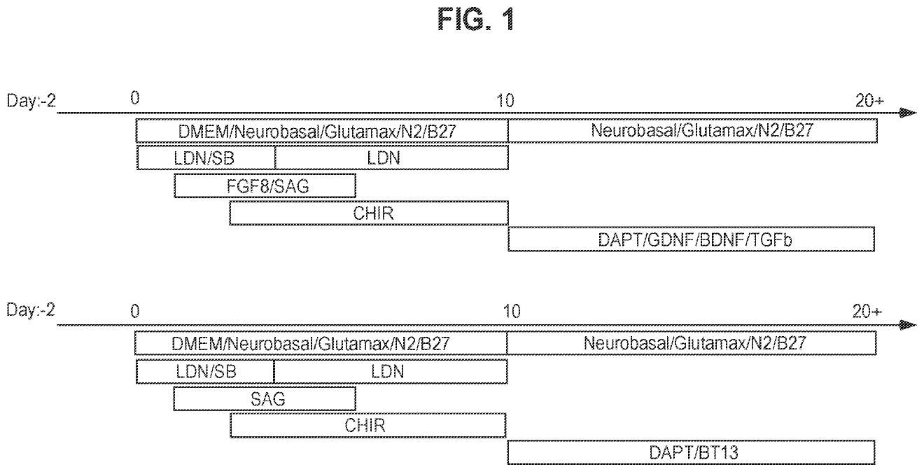

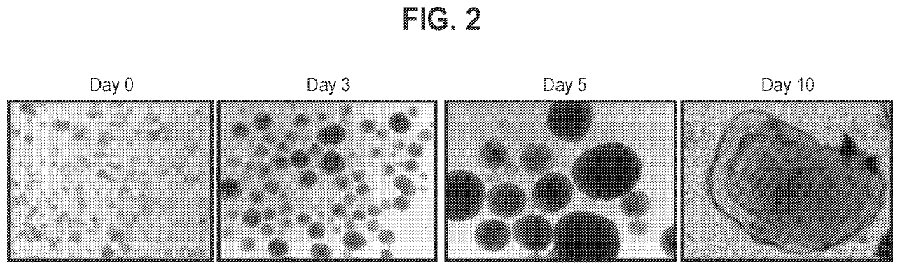

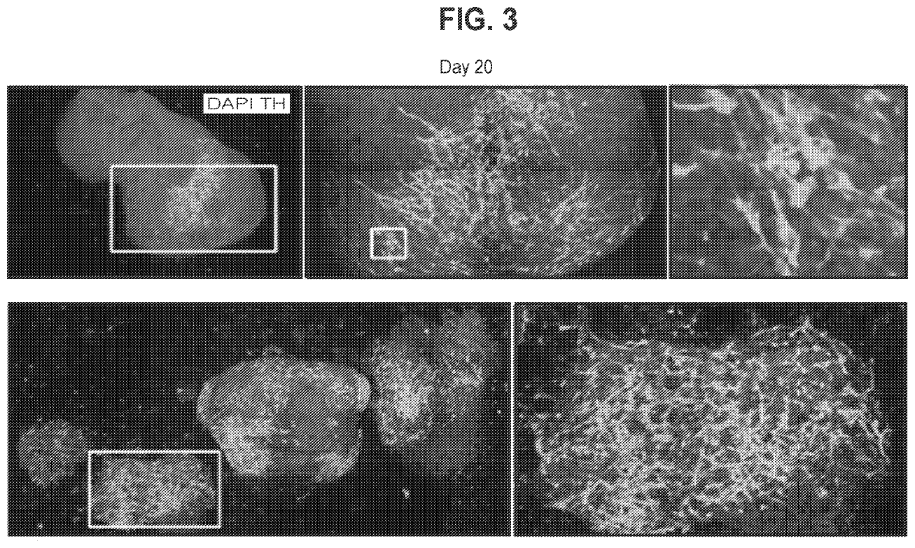

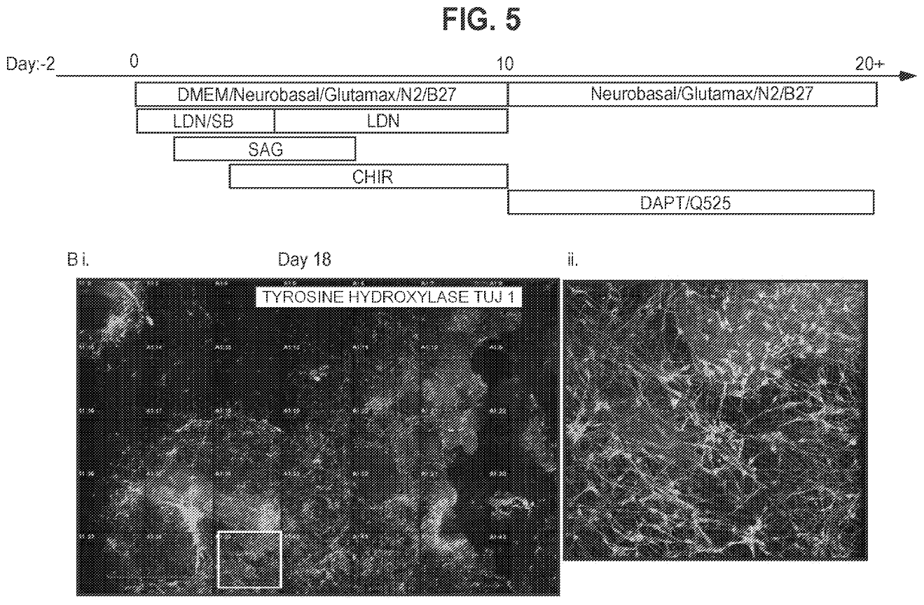

hPSC-derived dopaminergic neurons have demonstrated safety and efficacy as a cell therapeutic for Parkinson's disease in numerous rodent and non-human primate animal studies and human trials have been initiated. Due to their significant progress and promise for clinical translation, production of hPSC-derived dopaminergic neurons was selected as a critical use-case for a minimal protein media formulation whereby recombinant proteins FGF8, TGF-0, BDNF, and GDNF were removed and replaced with BT-13 or Q525 starting Day 10 (FIGS. 1 (BT-13) and 5 (Q525)) in a 3D differentiation system as described in Adil et al., Sci Rep, 7:40573 (2017). Removal of FGF8 during the first 6 days of differentiation did not impact the growth or patterning of the 3D aggregates (FIG. 2). By day 20, expression of tyrosine hydroxylase, the rate-limiting enzyme in dopamine production and functional biomarker of dopaminergic neurons, was detected in neuronal cell bodies within all of the differentiating aggregates (FIGS. 3 and 5). The replacement of GDNF, BDNF, and TGF-β by BT-13 or Q525 is applicable to all neuronal subtypes requiring GDNF for differentiation (see Table 1). The results presented herein demonstrate that two structurally unrelated RET agonists can each replace GDNF, BDNF and TGF-0 in the production of functional dopaminergic neurons from pluripotent cells and as such support the use of any GDNF receptor RET agonist in the methods herein described.

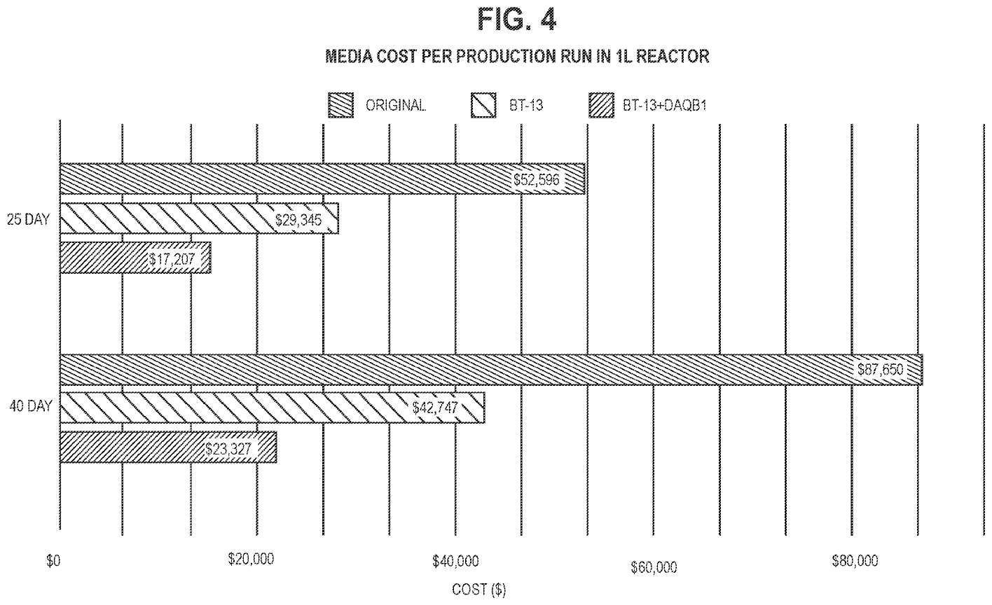

The cost of scaling up to a 1-liter bioreactor and adopting cGMP to manufacture hPSC-derived dopaminergic neurons for clinical development and commercialization was then modeled and the cost of using the original media formulation compared to the minimal protein BT-13 substitute media formulation was compared (FIG. 4). The stage of differentiation for optimal implantation and efficacy remains an area of active investigation; shorter differentiation time runs the risk of unpurified cell populations containing uncommitted proliferative precursors that may result in off-target differentiation or undesired ell overgrowth after implantation while longer differentiation time runs the risk of more mature and committed cells that are less resilient to the stresses of implantation and increased cell death during implantation. Therefore, we included two scenarios in the cost analysis: a 25-day differentiation for implantation of dopamine precursors and a 40-day differentiation for implantation of dopamine neuroblasts (FIG. 4) and the minimal protein formulation with BT-13 is able to reduce material costs by more than 50% in longer differentiation protocols. Use of small molecule replacement for GDNF reduces cost of media to produce hPSC-derived neurons by more than 50%.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention.

1. A method for producing a cell population comprising functional neuron cells, the method comprising a step of culturing an induced neuronal precursor cell in a differentiation cell culture medium comprising one or more glial derived neurotrophic factor (GDNF) receptor transmembrane receptor tyrosine kinase REarranged during Transfection (RET) agonists.

2. The method according to claim 1, wherein the GDNF receptor RET agonist is selected from DNSP-11, BT-18, BT-44, XIB4035, BT-13 and Q525.

3. The method according to claim 2, wherein the GDNF receptor RET agonist is BT-13 and/or Q525 and wherein BT-13 is present in the cell culture medium at a concentration of about 2 μM to 20 μM or about 5 μM, and/or wherein Q525 is present in the culture medium at a concentration of about 1 to 100 nM.

4. The method according to claim 1, wherein the differentiation cell culture medium does not comprise one or more of GDNF, brain-derived neurotrophic factor (BDNF) and TGFβ.

5. The method according to claim 4, wherein the differentiation cell culture medium is essentially free of proteins.

6. The method according to claim 1, wherein the differentiation cell culture medium comprises a neurobasal medium supplemented with N-2 and/or B-27 supplement.

7. The method according to claim 1, wherein the differentiation cell culture medium comprises neurobasal medium and an insulin receptor activator and does not comprise N-2 and/or B-27 supplement.

8. The method according to claim 7, wherein the insulin receptor activator is demethylasterriquinone B1 (DMAQ-B1).

9. The method according to claim 1, wherein the differentiation cell culture medium comprises a notch pathway inhibitor.

10. The method according to claim 9, wherein the notch pathway inhibitor is DAPT (N-[2S-(3,5-difluorophenyl)acetyl]-L-alanyl-2-phenyl-1,1-dimethylethyl ester-glycine) or dibutyryl cAMP (db-cAMP).

11. The method according to claim 1, wherein the induced neuronal precursor cell is cultured in differentiation medium for a period of from about 4 days to about 110 days, from about 4 to about 60 days, from about 5 days to about 40 days or from about 16 days to about 32 days.

12. The method according to claim 1, wherein the functional neuron cells are selected from dopamine precursors, dopamine neuroblasts, striatal neurons, neuroepithelial stem cells, GABAergic interneurons, cortical interneurons, cholinergic neurons, serotonin interneurons, cerebellar neurons, sensory neurons, and motor neurons.

13. The method according to claim 12, wherein the functional neuron cell is a dopaminergic neuron.

14. The method according to claim 13, wherein at least 50%, at least 60%, at least 70%, at least 80%, at least 90% or more of the cells in the produced cell population express tyrosine hydroxylase (TH).

15. The method according to claim 1, wherein the induced neuronal precursor cell is produced by culturing a mammalian pluripotent cell in an induction medium comprising an effective amount of an inhibitor of a Bone Morphogenetic Protein (BMP) signaling pathway and optionally an effective amount of an inhibitor of a TGFβ signaling pathway.

16. The method according to claim 15, wherein the inhibitor of a Bone Morphogenetic Protein (BMP) signaling pathway is LDN193189 and/or the inhibitor of a TGFβ signaling pathway is SB-431542.

17. The method according to claim 15, wherein the induction medium further comprises an activator of the WNT signaling pathway and/or an activator of sonic hedgehog (SHH), optionally wherein the activator of the WNT signaling pathway is GSK3 inhibitor, preferably CHIR99021 and/or wherein the activator of SHH is Smoothened Agonist (SAG).

18. (canceled)

19. The method according to claim 15, wherein the induction medium does not comprise an FGF receptor (FGFR) agonist and/or does comprise an inhibitor of TGFβ signaling, optionally wherein the induction medium does not comprise an FGF receptor (FGFR) agonist and does comprise an inhibitor of TGFβ signaling.

20. (canceled)

21. A method for producing a population comprising functional neuron cells, the method comprising (i) culturing mammalian pluripotent cell(s) in a neurobasal cell culture medium under conditions sufficient to produce a neuronal precursor cell population, optionally wherein the neurobasal cell culture medium comprises LDN193189, CHIR99021 and smoothened agonist and does not comprise FGF8 and (ii) culturing the produced neuronal precursor cell population in a neurobasal cell culture medium comprising an effective amount of a GDNF receptor RET agonist, and optionally a notch pathway inhibitor for a duration of time sufficient to produce a population of functional neurons, wherein the neurobasal cell culture medium does not comprise GDNF, BDNF and TGFβ.

22. (canceled)

23. A cell culture medium useful for producing functional neurons from neuronal precursor cells, wherein the cell culture medium is a protein-free neurobasal medium comprising a GDNF receptor RET agonist, preferably BT-13, and a notch pathway inhibitor, preferably DAPT and/or db-cAMP, wherein the culture medium does not comprise GDNF, BDNF and TGFβ.

24-29. (canceled)


SEQUENCE LISTING (1 sequence)

SEQ ID NO: 1 - protein, 11 aa, synthetic construct
PPEAPAEDRSL